(19) United States
(12) Patent Application Publication
(10) Pub. No.: US 2026/0106010 A1
(43) Pub. Date: Apr. 16, 2026

(54) COMPUTER-AIDED THERAPY FOR TREATING INSULIN RESISTANCE AND/OR RESTORING GLUCOSE HOMEOSTASIS IN A SUBJECT IN NEED

(71) Applicant: PALTECH, Paris (FR)

(72) Inventors: Charles-Henri MALBERT, Bruz (FR); Maurice Reginald ALLOUCHE, Paris (FR)

(73) Assignee: PALTECH, Paris (FR)

(21) Appl. No.: 19/116,580

(22) PCT Filed: Sep. 28, 2023
(86) PCT No.: PCT/EP2023/076969

§ 371 Date: Mar. 28, 2025

(30) Foreign Application Priority Data
Sep. 30, 2022 (WO) PCT/IB2022/000548

(51) Int. Cl. G16H 20/10; G16H 10/60; G16H 30/20; G16H 30/40; G16H 80/00

(52) U.S. Cl. CPC G16H 20/10; G16H 10/60; G16H 30/20; G16H 30/40; G16H 80/00

(57) ABSTRACT

System and a method for computer-aided therapy for treating type 2 diabetes, insulin resistance and/or restoring glucose homeostasis in a subject in need thereof, using a main frame that receives a subject profile; receives a set of images associated with the subject; processes PET and CT images for obtaining a 2D distribution of the quantitative density of the GLP-1r along the portal vein; and identifies a region with lower GLP-1r along the portal vein for which the values of the 2D distribution are less than a given threshold and for which the 2D distribution is less than or equal to a reference value; the threshold being based on the distribution of GLP-1r along the portal vein.

### FIELD OF THE INVENTION

The aim of the present application is, within a streamlined cloud-based workflow, (i) to present comprehensively the expression of peptidergic receptor in patient in need obtained by quantitative nuclear medicine imaging, (ii) to compute a target based on the low expression receptor areas suitable for assisting surgeons in laparoscopic guidance and (iii) to track the evolution of the therapy aiming at local increasing the density of peptidergic receptors. The invention translates into the design of a streamlined, cloud-based software suite interacting on the one side with a referent MD software and on the other side with a targeting computer software suitable for human surgical procedures. The cloud-based software suite is also capable to incorporate nuclear medicine assets such as those supplied by querying and retrieve from PACS computers.

### BACKGROUND OF THE INVENTION

### General Overview

The invention relates to cloud-based tools suitable for non-nuclear medicine specialist such as diabetologist or endocrinologist MD to extract relevant information from nuclear medicine quantitative data exhibiting receptor density. This information could be presented in such form that it could be a useful support for these professional to decide for the adequacy of molecular-based guided surgery. The same invention also transforms the quantitative data merged with anatomical images into meaningful information suitable for operating-room targeting computer.

Improvements in molecular imaging (Vaz, Oliveira, Herrmann, & Veit-Haibach, 2020) together with parallel iterative refinements in nuclear medicine scanners (Vandenberghe, Moskal, & Karp, 2020) supplied the medical community with important information on the density of a given receptor with millimetric precision. These information's are mostly used in the oncology field to improve precision during surgical tumor removal but due to limitations in handling these complex data, the full potential of image targeting surgery is not used. Furthermore, there is no clear attractivity for minimal resection within the scope of the potential risk as a consequence of partial tumoral resection (Zaccagna et al., 2021). On the contrary, an improved spatial precision for drug delivery is eagerly requested to reduce harmful side-effects of molecules with limited therapeutic index. The theranostic paradigm represents one successful attempt but is limited to therapies aiming at cell destruction (Hapuarachchige & Artemov, 2020).

To date the vast majority of non-irradiating targeted drug delivery was achieved using nanoparticle embedding of the active molecule using local administration of the nanosystem in the vascular bed to refrain its distribution body-wide (Paul & Sharma, 2020). This solution takes the advantage of the arterial tree to concentrate a given molecule in highly perfused organ such as the liver or the heart ventricle. Unfortunately, it is not suitable for functional structures that share identical vascularization characteristics as their dysfunctional counterpart. These cases require the direct placement of the drug during a preferentially minimally invasive surgical procedure. The size of the functional or anatomical structure requiring therapy dictates the mandatory use of image-based guidance such as during the placement of active stent device during arterial placement of such devices (Capodanno & Monte, 2020).

### Usage in the Field of Diabetes

Glucose homeostasis, especially after a meal, requires the knowledge of the future potential evolution of glycemia before the actual arrival of glucose in the blood stream. Such goal was completed by an early detection of glucose arrival before its passage through the liver and the delivery of this information to the brain which ultimately control pancreatic insulin secretion (Soty, Gautier-Stein, Rajas, & Mithieux, 2017). This scheme is altered in T2D patients with a blunting in the portal sensor that became unable to accurately detect post-prandial glucose arrival (Duca, Waise, Peppler, & Lam, 2021). This detection of glucose required a functional density of GLP-1 receptor along the length of the portal sensor (Burcelin, Da Costa, Drucker, & Thorens, 2001). T2D patients displayed a reduced expression of GLP-1r at the portal level as a consequence of greater insulin resistance (Malbert, Chauvin, Horowitz, & Jones, 2020b). One solution relates to the local delivery of a targeted drug aimed to increase GLP-1r density at the exact positioning of the defective portal sensor. However, since the portal sensor is extending within a narrow length, it is impossible to achieve such delivery without additional clues such as those supplied by nuclear medicine-based image set (Malbert, Chauvin, Horowitz, & Jones, 2020b). Unfortunately, the translation from these nuclear medicine data to the actual deposit of the drug capable to increase GLP-1r density is complex and, given these intricacies, achievable in very few research centers only. The aim of our invention is precisely to fill this gap between the imaging of the GLP-1r density to the targeted delivery of the therapeutic molecule at the portal sensor level.

### Limitations for a Low-density Receptor Based Targeted Surgery

### Functional Imaging in Clinical Practice

Current clinical practice mostly uses, for imaging the diabetic patient, tools aimed at evaluating the diabetic complications key organs such as contrary arteries, heart and its associated autonomic neuropathy, kidney and lower limb (Yang, Zhang, Wang, Hong, & Liu, 2019). Notably, most of these imaging approaches are not specific for diabetes. On the contrary, despite several attempts, some of which being extremely convincing in imaging insulitis for instance, molecular imaging is still in its infancy within the diabetologia community (Rastogi & Jain, 2016). This results in an overall lack of knowledge about what kind of imaging is suitable and ultimately a large uncertainty on the imaging prescription itself (Placido et al., 2017). Conversely, because it is seldom used, the nuclear medicine departments have no experience on PET imaging relevant to diabetes pathophysiology. Furthermore, the diabetologist has not the current skill nor the appropriate tools to interpret the complex results of the imaging trial. Finally, the overall cost of the nuclear medicine investigation required a precise identification of the patients suitable to undergo the appropriate examination based on quantitative insulin-glucose homeostasis measurements, also a tool and a procedure that were not currently available in clinical practice (Gordon, Elliott, Joshi, Williams, & Vela, 2020).

### Accessing Receptor Density

In current nuclear medicine practice, access to receptor density metric is uncommon with the notable exception of DatScan within the scope of parkinsonian syndromes (Grabher, 2019) or in research protocols. In the first case, this was made possible by the availability of an easy-to-use dedicated software DatQuant (Brogley, 2019). Aside from the dopamine transporter specific instance, quantitative measurement of the receptor density requires laborious calculation involving compartmental or spectral analyses (Innis et al., 2007). These analyses are classically not performed in clinical setting since the sole usage of receptor imaging is to facilitate surgical ablation of endocrine tumors i.e., targeting structures exhibiting high receptor expression. The massive increase in radioactive emission of such structures can now easily achieved using miniature radioactivity detectors held at the tip of a laparoscopic forceps (Pashazadeh & Friebe, 2020). On the contrary, we are targeting low receptor density structure that cannot be tracked directly by a hand-held radioactivity probe. Therefore, the present patent application described the tracking of pre-operatively build 3D computer representation of low-density receptor structures identified using classical PET imaging. Currently, there is no streamlined solution for laparoscopic targeting based on hybrid anatomical/molecular mapping whatever the organ or the targeting method. While tracking of the surgical instrument is an essential step towards the improvement of the surgical workflow by a comprehensive surgical landscape guidance system, such system is still in its infancy (Hartwig et al., 2020).

### SUMMARY OF THE INVENTION

The aim of the invention is to provide a system and a method for computer-aided therapy for treating insulin resistance and/or restoring glucose homeostasis in a subject in need.

According to a first aspect, the invention concerns a system for computer-aided therapy for treating type 2 diabetes, insulin resistance and/or restoring glucose homeostasis in a subject in need, comprising a main frame configured for performing the following steps:
a) receiving a subject profile created during a consultation of the subject by a MD at a first point of care, the subject profile comprising data useful for a therapy and the follow-up of the subject;
b) receiving a set of images associated with the subject, the set of images have been obtained at a second point of care, the set of images comprising a 3D computed tomography, CT, images of the abdomen of the subject including the pancreatic area and the portal vein of the subject; and a set of dynamic 3D positron emission tomography, PET, images of the abdomen after an intra veinous injection of a radiolabeled GLP-1r ligand, PET images comprising the positron imaging of the GLP-1r in the abdomen, the radiolabeled ligand is binding on GLP-1r;
c) processing the PET and CT images for obtaining a 2D distribution of the quantitative density of the GLP-1r along the portal vein;
d) identifying a region with lower GLP-1r along the portal vein for which the values of the 2D distribution are less than a given threshold and for which the 2D distribution is less than or equal to a reference value; the threshold being based on the distribution of GLP-1r along the portal vein.

The system of the invention is advantageously completed by the following features, taken alone or in any technically possible combination thereof:
the main frame is configured for performing the following steps
   e) determining from the CT image and the 2D distribution, a first mesh (PV') representing the portal vein (PV);
   f) determining from the CT image and the threshold a second mesh (DPS') identifying the region with lower GLP-1r along the portal vein, the threshold being applied on the CT image;
   g) determining from the first mesh and the second mesh a final 3D mesh (FM) wherein the second mesh is inside the first mesh.
the main frame is configured for performing the step of:
   h) determining a targeting solution by combining the final 3D mesh with the 3D CT image, the 3D CT image comprising several markers corresponding to externally applied fiducial marks the targeting solution comprising a 3D volumetric rendering comprising the 3D mesh of the portal vein coded with the density of the GLP-1r along the portal vein depending on the position relative to the threshold.
the threshold is the mean—2*SE of the entire 2D distribution of the quantitative density of the GLP-1r along the portal vein.
CT image and 3D volumetric rendering comprise images of external fiducial markers located on the subject skin, preferably at least three fiducial markers and more preferably four or five fiducial markers whose positions in space XYZ are sufficient to identify the three dimensions of the surgical space.
step c) comprises the sub steps of: extracting from the CT image, a portal vein volume of interest including the portal vein; obtaining from the PET images, a receptor density map quantifying indicator of the GLP-1r density in the abdomen; obtaining from the receptor density map and the portal vein volume of interest a 3D image quantifying the GLP-1r density in the portal vein volume of interest; obtaining a 2D distribution of the quantitative density of the GLP-1r density along the portal vein by projecting the 3D image along a center line of the second volume of interest.
the region with lower GLP-1r for which the 2D distribution is less than the threshold, is a defective portal sensor to be treated.
the main frame is configured for performing a step of extracting from the CT image a veinous branching location which is the location of the gastric, duodeno-pancreatic and splenic veins, a step of transferring to a first computing system of a MD such as a diabetologist or endocrinologist veinous branching location and the 2D distribution, so that a first computing system implements a step of displaying the 2D distribution along the veinous branching location to aid the MD to make a decision to process further the subject with a surgery procedure.
the main frame is configured for performing a step of transferring the targeting solution to a third computing system at an abdominal surgeon/department.
it comprises an abdominal surgeon department connected to the mainframe, the abdominal surgeon department being configured for performing the steps of
   i) providing at least one bioactive molecule upregulating the expression of GLP-1r, and
   ii) locally administering said at least one bioactive molecule to the portal defective portal sensor identified as the mesh.
it comprises a third point of care, preferably an abdominal surgeon/department, the third point of care being configured for performing a step of determining a customized targeting solution by obtaining from the targeting solution a surgical procedure for accessing the area represented by the 3D mesh within the spatial CT reference.
the surgical procedure includes laparoscopy, radiological or endoscopic solutions or a mixture of these.
it comprises a first computer system configured to be accessed by a MD such as a diabetologist or endocrinologist; a second computer system configured to be accessed by specialist of nuclear medicine; at least one a third computer to be accessed by an abdominal surgeon/department; the first, second and third computer being connected to the mainframe by means of a secure link.

According to a second aspect, the invention concerns a method for computer-aided therapy for treating type 2 diabetes, insulin resistance and/or restoring glucose homeostasis in a subject in need, wherein a main frame is configured for performing the following steps:
a) receiving a subject profile created during a consultation of the subject by a MD at a first point of care, the subject profile comprising data useful for a therapy and the follow-up of the subject;
b) receiving a set of images associated with the subject, the set of images have been obtained at a second point of care, the set of images comprising a 3D computed tomography, CT, images of the abdomen of the subject including the pancreatic area and the portal vein of the subject; and a set of dynamic 3D positron emission tomography, PET, images of the abdomen after an intra veinous injection of a radiolabeled GLP-1r ligand, PET images comprising the positron imaging of the GLP-1r in the abdomen;
c) processing the PET and CT images for obtaining a 2D distribution of the quantitative density of the GLP-1r along the portal vein;
d) identifying a region with lower GLP-1r along the portal vein for which the values of the 2D distribution are less than a given threshold and for which the 2D distribution is less than or equal to a reference value; the threshold being based on the distribution of GLP-1r along the portal vein.

The method of the invention is advantageously completed by the following features, taken alone or in any technically possible combination thereof:
the main frame is configured for performing the following steps
   e) determining from the CT image and the 2D distribution, a first mesh (PV') representing the portal vein (PV);
   f) determining from the CT image and the threshold a second mesh (DPS') identifying the region with lower GLP-1r along the portal vein, the threshold being applied on the CT image;
   g) determining from the first mesh and the second mesh a final 3D mesh (FM) wherein the second mesh is inside the first mesh.
the main frame is configured for performing the step of: h) determining a targeting solution by combining the final 3D mesh with the 3D CT image, the 3D CT image comprising several markers corresponding to externally applied fiducial marks the targeting solution comprising a 3D volumetric rendering comprising the 3D mesh of the portal vein coded with the density of the GLP-1r along the portal vein depending on the position relative to the threshold.
the threshold is the mean—2*SE of the entire 2D distribution of the quantitative density of the GLP-1r along the portal vein.
CT image and 3D volumetric rendering comprise images of external fiducial markers located on the subject skin, preferably at least three fiducial markers and more preferably four or five fiducial markers whose positions in space XYZ are sufficient to identify the three dimensions of the surgical space.
step c) comprises the sub steps of: extracting from the CT image, a portal vein volume of interest including the portal vein; obtaining from the PET images, a receptor density map quantifying indicator of the GLP-1r density in the abdomen; obtaining from the receptor density map and the portal vein volume of interest a 3D image quantifying the GLP-1r density in the portal vein volume of interest; obtaining a 2D distribution of the quantitative density of the GLP-1r density along the portal vein by projecting the 3D image along a center line of the second volume of interest.
the region with lower GLP-1r for which the 2D distribution is less than the threshold, is a defective portal sensor.
the main frame is configured for performing a step of extracting from the CT image a veinous branching location which is the location of the gastric, duodeno-pancreatic and splenic veins, a step of transferring to a first computing system of a MD such as a diabetologist or endocrinologist veinous branching location and the 2D distribution, so that a first computing system implements a step of displaying the 2D distribution along the veinous branching location to aid the MD to make a decision to process further the subject with a surgery procedure.
the main frame is configured for performing a step of transferring the targeting solution to a third computing system at an abdominal surgeon/department.
an abdominal surgeon department is connected to the mainframe, the abdominal surgeon department being configured for performing the steps of
   i) providing at least one bioactive molecule upregulating the expression of GLP-1r, and
   ii) locally administering said at least one bioactive molecule to the portal defective portal sensor identified as the mesh.
a third point of care, preferably an abdominal surgeon/department is configured for performing a step of determining a customized targeting solution by obtaining, from the targeting solution a surgical procedure for accessing the area represented by the 3D mesh within the spatial CT reference.
the surgical procedure includes laparoscopy, radiological or endoscopic solutions or a mixture of these.

The targeting solution combined the threshold and a binarized version of the GLP-1r density, together with externally applied fiducial marks referenced with the CT image, for obtaining a 3D volumetric rendering comprising a 3D mesh of the portal vein coded with the density of the GLP-1r along the portal vein depending on the position relative to the threshold.

The workflow of the invention is preferably designed to supply a patient follow-up from its initial pathological condition towards the final cure. This is, in particular, achieved using a software interface dedicated to the diabetologist/endocrinologist that depicts the current patient status based on various exams including nuclear medicine imaging of the portal receptor density. This software interface will exchange seemingly data with a cloud-based evaluation and targeting processor. This processor will in turn supply targeting solution to the laparoscopic surgeon after the upload of the targeting solution into the targeting apparatus to be used during the surgical procedure. The procedure will be used to insert at a precise location defined by a lower expression of peptidergic receptors within the portal system either the tip of a surgical tool suitable to insert slow-release pellets or other slow-release pharmaceutical form of a given compound suitable to increase the expression of peptidergic receptors.

The invention is preferably designed to accommodate several radioligands that share the same peptide agonist moiety but with different linker molecules between the radio-isotope itself and the receptor specific moiety resulting in different pharmakinetics properties towards the receptor expressing abdominal organs namely the pancreas and the duodenum that express large quantity of GLP-1r for example. This is achieved by a complex yet robust high-density to low-density meshes reconstruction centered on the portal vein.

### BRIEF DESCRIPTION OF THE DRAWINGS

Other features and advantages of the invention will appear in the following description. Embodiments of the invention will be described with reference to the drawings:

FIG. 1 illustrates an architecture for implementing a method for computer-aided therapy for treating diabetes type 2, insulin resistance and/or restoring glucose homeostasis in a subject/patient in need according to the invention.

FIG. 2 illustrates the steps of a method for computer-aided therapy for treating insulin resistance and/or restoring glucose homeostasis in a subject in need according to an embodiment of the invention implemented in the architecture illustrated on FIG. 1.

FIG. 3 illustrates the steps relating to the processing of the nuclear images implemented by the preprocessing unit of the architecture illustrated on FIG. 1.

FIG. 4 illustrates the importance and the effects of the radioligand molecular structure on the GLP-1r pancreatic but not portal binding demonstrating the need for a portal clue for computing thresholding.

FIG. 5 illustrates two typical examples of the GLP-1r density (Vs (mL/cc m) along the portal vein (abscissas) depending on the patient condition: lean insulin sensitive and type 2 diabetes.

FIG. 6 illustrates the steps for computing the portal high density and low density meshes especially used by the targeting computer during the surgical procedure.

FIG. 7 illustrates one and example, here using optical tracking, of a laparoscopic tool navigated using real time 3D navigation.

### DETAILED DESCRIPTION OF THE INVENTION

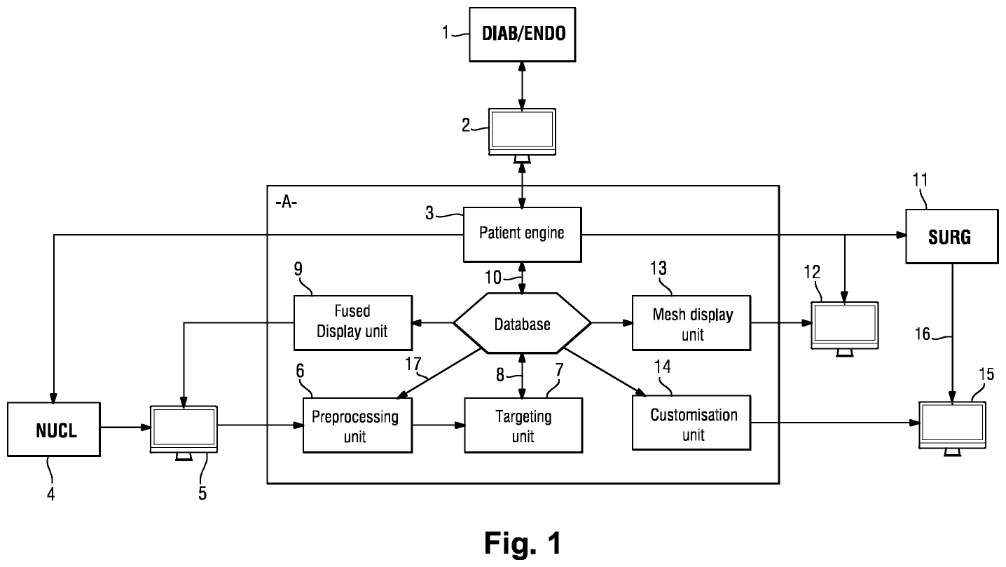

FIG. 1 illustrates an architecture for implementing a method for computer-aided therapy for treating insulin resistance and/or restoring glucose homeostasis in a subject/patient in need. This method involves a MD such as diabetologist or endocrinologist DIA/END 1, a nuclear physician NUCL 3 and an abdominal surgeon, an intervention radiologist or a gastroenterologist, the term surgeon SURG 11 will be used in the following. This architecture permits to have tools suitable for the non-nuclear medicine specialist i.e., the diabetologist or endocrinologist MD to obtain relevant information from nuclear medicine through the quantitative data exhibiting receptor density. It also translates the target into workable mesh superimposed with computed tomography (CT) image suitable for live interaction with the surgeon in the operating theatre.

This illustrated architecture is centered on a mainframe A preferably a remote secured mainframe A which is designed to supply a patient follow-up from its initial pathological condition towards the final cure. The mainframe A is in particular configured to process data and comprises one or more dedicated processor and one or more database.

The method starts by the interaction of the patient in need and the diabetologist 1 who via a simplified interface 2 connected to the main frame A introduces patient details. The simplified interface 2 is for instance a computing system. During the entire method, data of the patient in need are identified under a unique identifier generated by the MD when the patient information is initially introduced in the simplified interface 2.

These details are thus available, preferably automatically, to the nuclear physician NUCL 4 and to the surgeon SURG 11 by means of interfaces 5, 12, 15 such as a computing system. This link allows the nuclear physician NUCL 4 to create a required appointment for acquisition of images and for PET-CT imaging (Positron Emission Tomography-CT). According to an embodiment, the MD with the simplified interface 2 is able to directly generate a rendezvous to the nuclear medicine department and to a surgical structure if needed.

The nuclear medicine department has preferably PACS DICOM (Picture Archiving Communication System) communication capabilities. Once the images are acquired and made available on the PACS server of the nuclear medicine department on the nuclear physician interface 5 (i.e., a computing system), the acquired images are sent to the main frame A through a communication link (e.g., Internet link). At the main frame A the raw images are thus received.

The main frame A aims, in particular, at processing the images. The image processing comprises several steps implemented by the main frame A.

A preprocessing unit 6 receives the images and is configured to transform the raw dynamic data into receptor density coded image suitable for further processing. This newly coded image is then transferred to a targeting unit 7 to pinpoint the area where the surgeon SURG 11 will have to implant the bioactive molecule during the surgery. The information including the raw images, the receptor density converted images and the targeting data is stored in a comprehensive database 8 that could be a Structured Query Language (SQL) database with interoperation scheme using JDBC interfaces between functional units. Introduction of new complete dataset into the database 8 informs a patient engine 10 which in turn elicit connection with the nuclear physician who will be able to write his report based on the processed data. The patient engine 10 is in particular configured to manage the anonymization of the patient data and their format.

These processed data are comprehensively presented to the nuclear physician interface 5 using a fused display unit 9 connected to the database 8. Indeed, the fused display unit 9 is configured to format the image data in order to be transferred to nuclear physician interface 5.

Data originating from the nuclear medicine department and back to the nuclear medicine department after their processing at the main frame A are preferably coded in DICOM and the links are also generated through DICOM standard which is a well-known storage for this type of image.

Upon arrival of completed data together with the report from the nuclear physician and after an approval by the MD, the diabetologist for instance, for continuation of the procedure, a 3D-mesh suitable for fusion with CT image is created at the main frame A by means of a mesh display unit 13. This mesh together with the other images are made visible on a surgical planning computer 12 that is located outside the surgical theatre so that the surgeon 11 has the capability to optimise the access path to the area of interest represented by the mesh. Once the surgeon 11, the intervention radiologist or the gastroenterologist has designed an access path using 3D virtual representation of subject, the main frame A further processes the data present in the database 8 to adapt the targeting to the specificity of the surgical speciality. This is achieved by a customisation unit 14. This customisation unit is therefore dependant on the speciality of the surgeon. It has also the capability to upload the information required for live targeting in the surgical theatre into a dedicated targeting device 15 allowing surgeon interaction during the procedure. This dedicated targeting device is for instance a computer.

According to an embodiment, the main frame A is also configured to build Al based interaction between the database 8 and the processing units 6, 7 using the data already present and analysed as source for self-learning. This process is exemplified through the interaction between the database and the preprocessing unit 6 and the targeting unit 7.

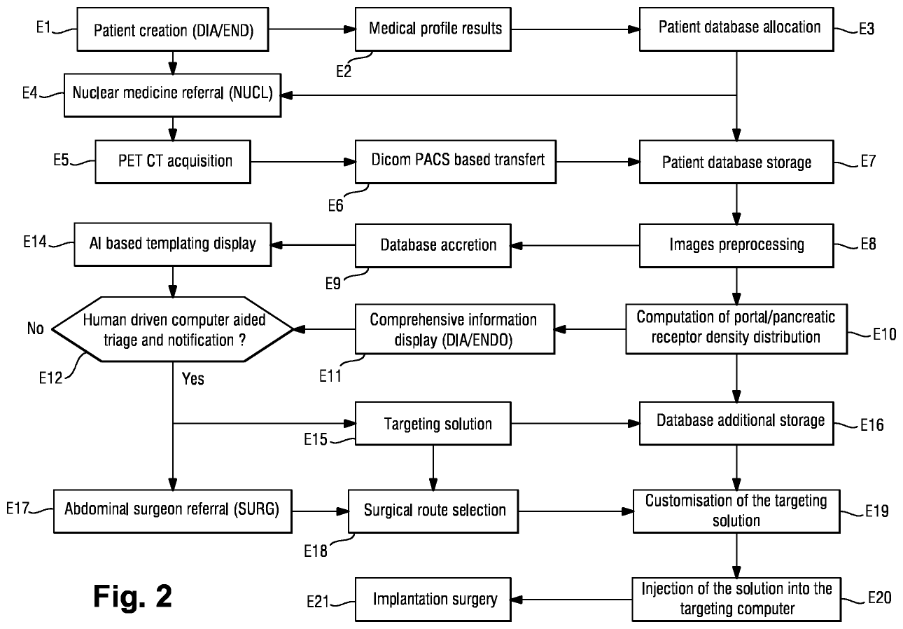

FIG. 2 illustrates steps of a method for computer-aided therapy for treating insulin resistance and/or restoring glucose homeostasis in a subject in need according to an embodiment invention implemented in the architecture previously described.

A MD preferably a diabetologist or endocrinologist DIA/ENDO creates a patient file with patient details by means of the simplified interface 2 (step E1). This patient/subject file is created during a consultation of the patient in need preferably using the classical rules for patient anonymization across Internet link. This patient/subject file provides all the patient's data useful for the treatment and the follow-up of the patient (step E2). The patient/subject file is then transferred from the simplified interface 2 to the mainframe A and is stored in the database 10 (step E3) through the patient engine 3 interfacing the simplified interface 2 with the database 8.

Thus, the nuclear physician 4 has access to all the information of the patient coming from the diabetologist 1 through the main frame A. Simultaneously, preferably, based on the nuclear medicine referral favorite included in the simplified interface 2, the patient is referred to a nuclear physician 4 together with all the required analysis profile (step E4).

The nuclear physician 4 at the nuclear department acquires (step E5) images of the same region of the patient.

The nuclear images are preferably dynamic 3D positron emission tomography (PET) images acquired after IV injection of a radiolabeled agonist such as [68Ga] Ga-NODAGA-Exendin-4 for GLP-1r imaging.

The CT image permits to show a general view of a region of interest including the portal vein, the head and the tail of the pancreas, the stomach and the liver while the nuclear images show a quantitative indicator of the receptor density in that same region.

The obtention of the nuclear images (dynamic PET and CT images) are well known by the man skilled in the art.

Following their acquisition, these images are transferred to the main frame A (step E6) by means of the nuclear physician interface 5 which is has PACS DICOM communication capabilities and of which the receiver characteristics were supplied in the referral parameters for configuring the communications between the involved physicians. The image data together with the images are then stored in the database 8 (step E7).

The images previously stored and coming from the database 8 are then processed by the preprocessing unit 3 during a preprocessing step (step E8) for obtaining
a receptor density map (vs) quantifying indicator of the GLP-1r density in the abdomen;
a 3D image quantifying the GLP-1r density in the portal vein volume. Vs is in particular the distribution volume of the GLP-1r i.e., the slope of the linear regression to Logan plot from t* (see Logan et al.).

These results are then stored in the patient database 8 (step E9).

The preprocessing unit 3 during the preprocessing step (step E8) combines the information coming from the CT image and from the PET images for obtaining 3D image quantifying the GLP-1r density in the portal vein volume. Then, this 3D image is processed for obtaining a 2D distribution of the quantitative density of the GLP-1r density along the portal vein (step E10). Additionally, according to an embodiment this distribution is compared to a threshold (but for some radioligand only—probably not the one we will be using Ga-NODAGA-Exendin 4).

According to an embodiment the preprocessing step results in a comprehensive graphical information suitable for display on a non-image specialist which is in the present invention a diabetologist or endocrinologist (step E11).

This display is presented to the diabetologist or endocrinologist who will transmit information of the patient into a medical workflow based on a computer aided triage and notification process (step E12), in particular for notifying and routing the patient to the relevant specialist.

According to an embodiment, results obtained during the preprocessing step are stored into a database for artificial intelligence network learning to achieve, if possible, automatic detection of the optimal placement of the therapeutic device e.g., the area with the lower GLP-1r expression (step E14).

The 2D distribution permits to identify areas of low and high GLP-1r density by a comparison with a threshold. In particular, a defective portal sensor (i.e., the area with the lower GLP-1r expression) is located which is a region with lower GLP-1r for which the 2D distribution is less than threshold.

In particular, this distribution permits to obtain a targeting solution (step E15) consisting in a 3D image comprising a mesh of the portal vein color coded with the density of the receptor density depending on the position relative to the threshold level.

The targeting solution is advantageously encapsulated into a comprehensive file package and stored in the database 8 (step E16).

After acknowledgment for continuation of the procedure by diabetologist or endocrinologist, the patient is referred to an abdominal surgeon/department (step E17).

This is achieved using the referral favorite surgeon included in the simplified interface 2 at the diabetologist side. The surgeon 11 has access of all information stored in the main frame A, in the database 5 with preferential access to the targeting solution. Using all these information, the surgeon selects the optimal surgical procedure approach based on the available targeting solution (step E18). These included laparoscopy, radiological or endoscopic solutions or a mixture of these. Because of the specificities of these procedures, the targeting solution needs to be adapted in a customization step before being used in the surgical theatre (step E19). After this adaptation, the customised targeting solution is automatically downloaded to the targeting computer 15 preferably using a secure internet channel without human interaction (step 20). The surgeon uses the targeting computer 15 once adequately configured with the patient map to complete the surgical implantation of the therapeutic device (step E21).

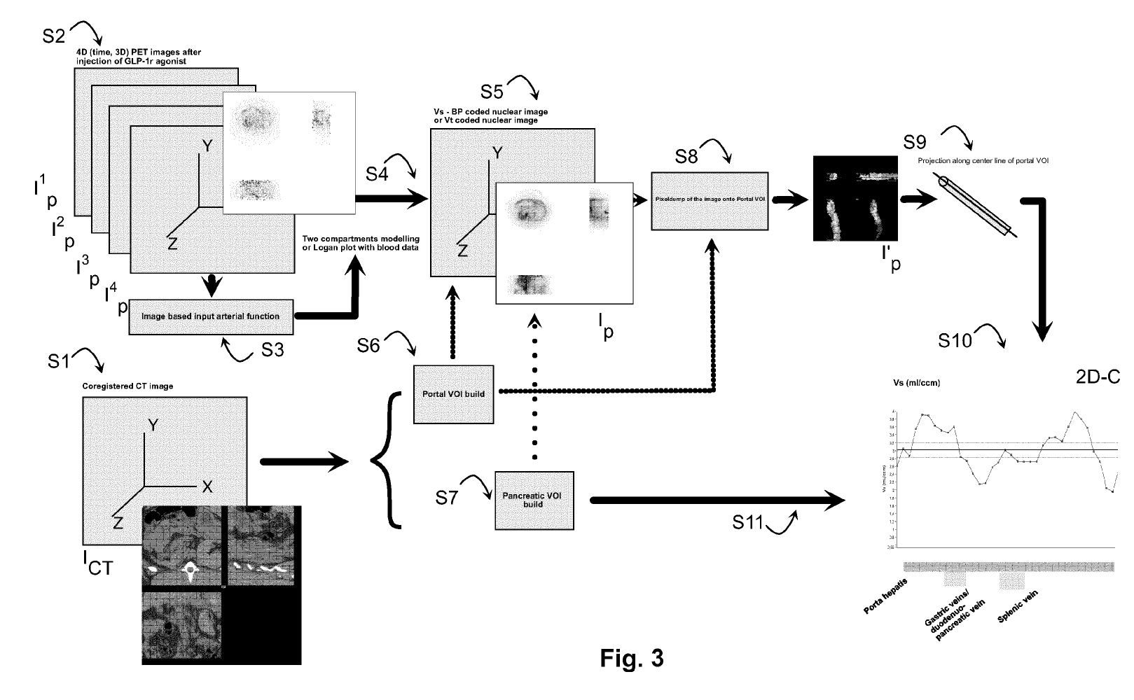

FIG. 3 describes the steps related to the processing of the nuclear images implemented by the preprocessing unit 6. As already mentioned, these steps are parts of the method implemented in the architecture described in FIG. 1.

First, a CT volume I_{CT}, including the portal vein is obtained (step S1). From this volume, two volumes of interest are defined (steps S6 and S7): a first volume of interest engulfed the pancreas (Pancreatic VOI) and a second volume of interest (Portal VOI) included the portal vein.

Scone, a set of 3D images I¹P, I²P, I³P, I⁴P acquired in dynamic mode with time as 4th dimension (step S2) and originating from the positron imaging (PET) of the GLP1r is also obtained. In a preferred embodiment, the dynamic frame sequence of PET acquisition be for 25 min is constituted by 30 frames: (12×10s, 6×30s, 5×20s, 2×300s).

From this 4D set of PET images, a volume of interest defining the abdominal aorta is obtained and used to extract the arterial input function (step S3). The extraction of the input function also includes partial volume correction according to (Rousset et al.) Once extracted, the corresponding function is transformed into plasma based arterial input function suitable for the remaining of the processing. This is achieved by multiplying individual values by the plasma/blood radioactivity ratio obtained after discrete blood sampling during nuclear image acquisition. In the absence of discrete blood sampling, a population-based distribution is used.

A Vs-Binding potential coded image Ip of the abdomen (step S5) is calculated voxel wise using a two compartmental modelling (step S4) with the corrected arterial input function (step S3) and the set of PET images. Alternatively, to two compartment modelling, the Vs-Binding potential coded image is obtained using a Logan plot (Logan et al.) The Vs-Binding potential image is a 3D coded nuclear image representing the density of the GLP-1r. This Vs-Binding potential image Ip serves as an initial step to extract the quantitative density of GLP-1r along the portal vein I'_{P} (step S8) using the portal VOI build previously obtained.

Preferably, pixel dump of the values circumscribed by the portal VOI is exported in a matrix format, including for each incremental Z, all the values for total X and Y. Incrementation in XY and Z directions are those defined by pixel incrementation. A curve 2D-C representing the Vs/Binding potential along the portal vein, is obtained (step S10) using projection of this matrix along the center line of the portal VOI (step S9).

The curve 2D-C serves for identifying the region with lower GLP-1r expression along the portal vein which is obtained after the comparison of the distribution 2D-C with a threshold.

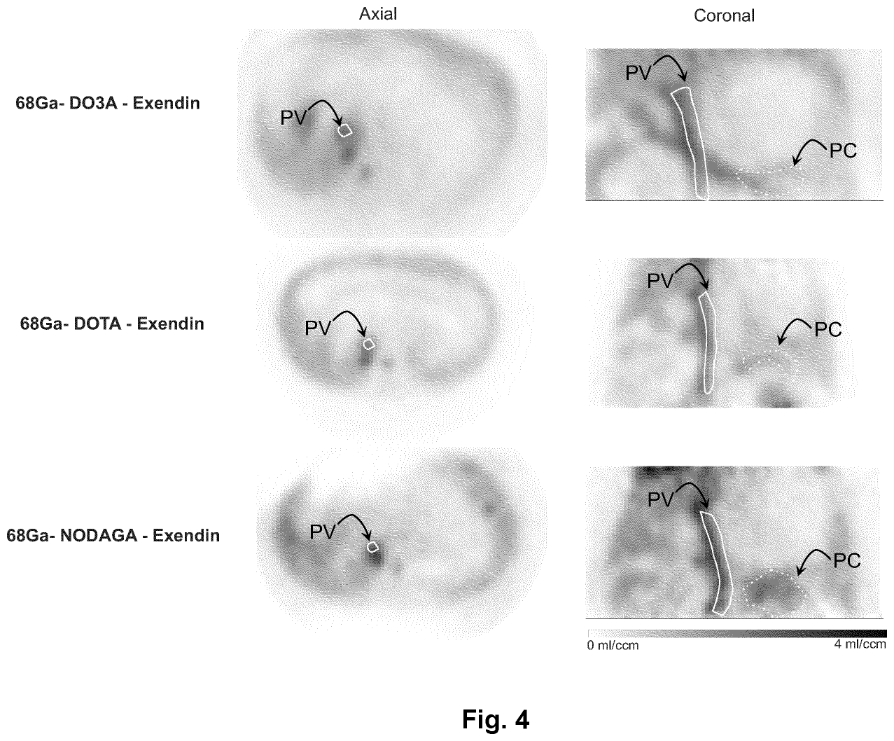

The value of Vs (which is an absolute value) at the portal VOI is highly dependent of the molecular composition of the radioligand despite sharing the same receptor binding moiety (e.g., Exendin 4) as illustrated on FIG. 4.

FIG. 4 shows examples of PET Vs coded images centered on the portal vein (underlined by a line PV which itself represents the portal VOI). The dotted line PC underlines the pancreas VOI. This figure demonstrates that the ratio between portal Vs and pancreas Vs depends on the chemical nature of the radioligand: 68Ga-DOTA-Exendin being the most extreme example with extremely low pancreas Vs while on the opposite 68Ga-NODAGA-Exendin generates relatively important pancreas Vs. The variation in Vs values of the pancreas (as obtained using S11) relative to portal vein makes impossible to extract a universal threshold from the intra-individual mean pancreatic Vs especially since in clinical practice numerous radioligand could be used.

To overcome this, an algorithmic strategy permits to extract a meaningful threshold from the 2D-C curve.

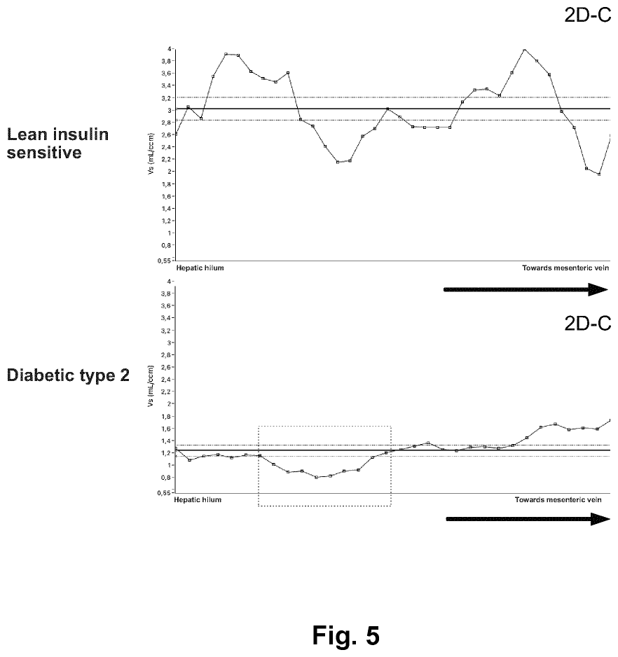

FIG. 5 summarizes the algorithmic strategy used to compute the threshold for two examples; one being lean insulin sensitive without therapeutic need the other being type 2 diabetic with the therapeutic need of a surgical targeting solution.

The 2D distribution permits to identify a defective portal sensor (i.e., the area with the lower GLP-1r expression) which is a region with lower GLP-1r for which the 2D distribution is less than the threshold.

First, the 2D distribution is examined and is processed only if it comprises value of Vs less than or equal to a reference value of Vs. This value is chosen such as to precisely target the defective portal sensor. The inventors have identified that a reference value of Vs equal to 1 permit to target this defective portal sensor. This reference value is chosen for discarding portal veins of healthy patient and for leaving only the patient in need.

One can note that a reference value of 1 is a very conservative criterion since it appears unlikely that the concentration of specifically bound radioligand (Cs) was less than that of the radioligand in the plasma (Cp) at equilibrium (Vs=Cs/Cp, see Innis et al, 2007). FIG. 5, presenting examples of lean insulin sensitive vs type 2 diabetic portal Vs profile, shows the magnitude of the difference between the two conditions.

Once the 2D distribution comprises values having value of Vs less than or equal to the reference value of Vs, the mean±2*SE (SE is the well-known standard error) is computed for the entire portal cross sectional profile. The value of Vs lower or equal to the reference value of Vs (1 for FIG. 5) and less than or equal to the mean—2SE of all the values permits to locate the defective portal sensor (framed on FIG. 5) represents the threshold for the remaining of the process (mean is represented as a solid line and the 2*SE value is represented as dashed lines). Note that the point that crosses the cross-sectional profile is not representative of the cut-off between the low and high expression segment since it will be calculated woxel wise for all the portal VOI (yet based on the above calculated threshold).

The defective portal sensor is thus the region for which the Vs values are:
less than or equal to the reference value of Vs (for discarding healthy portal vein) (e.g., 1); and
less than or equal to the mean −2SE computed for the entire portal cross sectional profile.

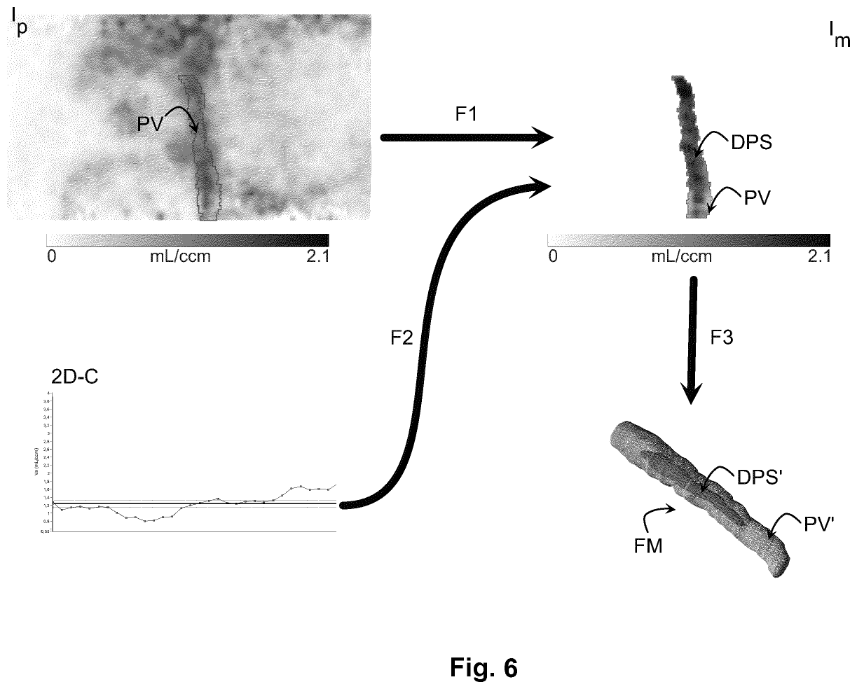

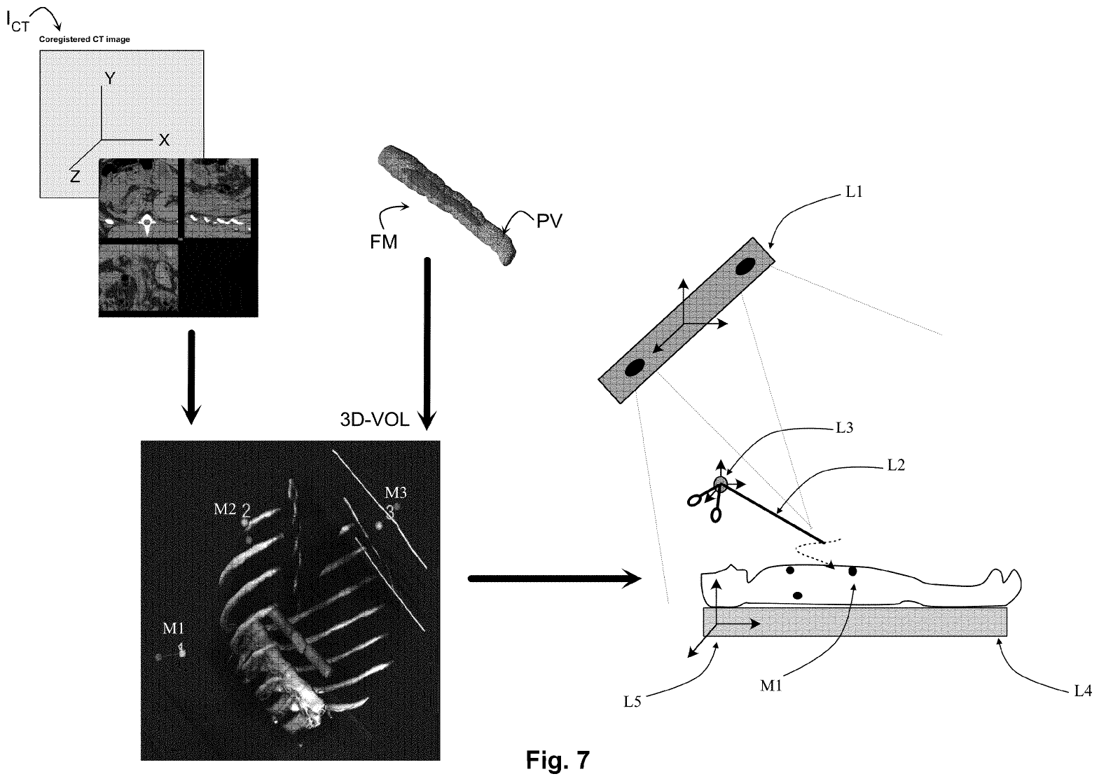

The threshold is then applied voxel-wise onto the Vs coded image (Ip) to create mesh files representing either the low-density or the high-density receptor structure. The process to build such meshes is described in FIG. 6. The voxels comprised in the portal VOI PV are preserved (step F1) while those outside the VOI are masked with zero value resulting in an image Im. Image Im is afterwards thresholded with mean±2 SE previously obtained in FIG. 5 (step F2). This process (step F2) is based on isocountouring (according to Lee et al, 2016) resulting in a second VOI representing the low-density segment creation for the defective portal sensor DPS. The VOI's are afterwards transformed in a final 3D mesh FM (step F3) within the same volumetric reference than the Vs coded image Ip (FIG. 6, right bottom plane). This final 3D mesh thus comprises the mesh representing the portal vein (mesh PV′) and the mesh representing the defective portal sensor (mesh DPS′). This 3D mesh is preferably recorded internally in vtk or in stl format suitable for display on a 3D volumetric rendering software superimposed with the coregistered CT and defined a targeting solution. This 3D representation including the final mesh is exported to the fusion computer for surgeon advice and ultimately to the targeting computer used in the surgical theatre as depicted in FIG. 7.

The final 3D mesh FM is advantageously displayed on a 3D volumetric rendering 3D-VOL comprising several markers (indicated M1, M2, M3 in FIG. 7). The final 3D mesh along with the 3D volumetric rendering 3D-vol defines the targeting solution.

The markers correspond to external fiducial markers located on the patient skin and are acquired during the CT image acquisition. These markers are mandatory to perform surgical navigation. At least three markers are necessary, but four or five markers are preferably used for improving precision.

Markers for CT scan are plastic coins glued to the patient skin and incorporating a small ball bearing component of 1 mm diameter for instance. The position of this marker is also pin pointed on the patient skin using surgical grade pen so to be able to identify it while the plastic coins is removed at the completion of the CT scan. These markers have the same tridimensional reference as the one used for mesh depiction and location of external fiducial markers located on the patient skin and visible both on CT scan and direct vision. The position of these markers will be made corresponding between their actual position on the patient and their relative position in the 3D-VOL so to co-reference the virtual representation 3D-VOL to the real (patient) spatial space in the surgery theatre accordingly to FIG. 7.

FIG. 7 also represents one example of surgical real time tool navigation within the patient space using a 3D optical navigation system (L1). This navigation system is only presented as an example since alternates systems may be equally used e.g., electromagnetic navigation system.

First co-registration between the patient space and the CT/mesh space is achieved by pin-pointing each of the markers in both spaces using a navigated tool (L2) on the one-hand and the virtual marker present on 3D-VOL (M1, M2, M3). Once these points were identified in both spaces, a rigid transformation (for correspondence of the two 3D matrices through a computed third 3D matrix which will be applied continuously to the optical acquired signal) is performed by the surgical computer resulting in actual tracking in one single space—the patient space. To achieve these steps, the 3D optical localizer (L1) tracks continuously a 3D frame (L3), unique in its design and attached to the surgical localizer (L2) which is suitable to be inserted through a laparoscopic canula. The detection of this 3D frame (L3) by the 3D camera localizer supplies to the surgical computer the position of surgical localizer (L2). Relative motion of the surgical table (L4, and the patient lying on it) against the optical localizer is cancelled using a third localizer (reference, L5) directly attached to the table so the surgical computer tracks continuously both the reference localizer and the surgical localizer. Permanent difference calculation between the position of both localizers (L3−L5) allows motion table free artifacts.

### REFERENCES

Brogley, J. E. (2019). DaTQUANT: The Future of Diagnosing Parkinson Disease. Journal of Nuclear Medicine Technology, 47(1), 21-26.

Burcelin, R., Da Costa, A., Drucker, D., & Thorens, B. (2001). Glucose competence of the hepatoportal vein sensor requires the presence of an activated glucagon-like peptide-1 receptor. Diabetes, 50(8), 1720-1728.

Capodanno, D., & Monte, M. A. (2020). Stent Wars. JACC: Cardiovascular Interventions, 13(14), 1706-1708.

Duca, F. A., Waise, T. M. Z., Peppler, W. T., & Lam, T. K. T. (2021). The metabolic impact of small intestinal nutrient sensing. Nat Commun, 12(1), 903.

Gordon, L. G., Elliott, T. M., Joshi, A., Williams, E. D., & Vela, I. (2020). Exploratory cost-effectiveness analysis of 68Gallium-PSMA PET/MRI-based imaging in patients with biochemical recurrence of prostate cancer. Clin Exp Metastasis, 37(2), 305-312. Grabher, B. J. (2019). DaTscan Imaging. Journal of nuclear medicine technology. Hapuarachchige, S., & Artemov, D. (2020). Theranostic Pretargeting Drug Delivery and Imaging Platforms in Cancer Precision Medicine. Front Oncol, 10, 1131. Hartwig, R., Ostler, D., Feußner, H., Berlet, M., Yu, K., Rosenthal, J.-C., & Wilhelm, D. (2020). COMPASS: localization in laparoscopic visceral surgery. Current Directions in Biomedical Engineering, 6(1).

Innis, R. B., Cunningham, V. J., Delforge, J., Fujita, M., Gjedde, A., Gunn, R. N., etc. Ichise, M. (2007). Consensus nomenclature for in vivo imaging of reversibly binding radioligands. Journal of Cerebral Blood Flow & Metabolism, 27(9), 1533-1539. Lee H, Paeng J C, Hong S H, Yoo H J, Cheon G J, Lee D S, Chung J K, Kang K W: Appropriate margin thresholds for isocontour metabolic volumetry of fluorine-18 fluorodeoxyglucose PET in sarcoma: a hybrid PET/MRI study. Nucl Med Commun 37:1088-1094, 2016.

Logan J, Fowler J S, Volkow, etc. ND: Graphical Analysis of Reversible Radioligand Binding from Time—Activity Measurements Applied to [N-¹¹C-methyl]-(-)-Cocaine PET Studies in Human Subjects. Journal of Cerebral Blood Flow and Metabolism 10:740-747, 1990.

Malbert, C. H., Chauvin, A., Horowitz, M., & Jones, K. L. (2020b). Pancreatic GLP-1r binding potential is reduced in insulin-resistant pigs. BMJ Open Diabetes Res Care, 8(2).

Malbert, C.-H., Chauvin, A., Horowitz, M., & Jones, K. L. (2020b). Glucose-Sensing Mediated by Portal GLP-1 Receptor is Markedly Impaired in Insulin-Resistant Obese Animals. Diabetes.

Pashazadeh, A., & Friebe, M. (2020). Radioguided surgery: physical principles and an update on technological developments. Biomed Tech (Berl), 65(1), 1-10.

Paul, W., & Sharma, C. P. (2020). Inorganic nanoparticles for targeted drug delivery. In Biointegration of Medical Implant Materials (pp. 333-373). Elsevier.

Placido, R., Calcaterra, D., Canitano, S., Capodieci, G., Di Modica, G., Marino, M. A., Orlacchio, A. (2017). COLLABORADI: a rule-based diagnostic imaging prescription system to help the general practitioner to choose the most appropriate radiological imaging procedures. Radiol Med, 122(3), 186-193.

Rastogi, R., & Jain, S. K. (2016). Imaging in diabetes mellitus. Arch Clin Nephrol 2(1): 017-025.

Rousset OG, Collins DL, Rahmim A, Wong DF: Design and Implementation of an Automated Partial Volume Correction in PET: Application to Dopamine Receptor Quantification in the Normal Human Striatum. Journal of Nuclear Medicine 49:1097-1106, 200

Soty, M., Gautier-Stein, A., Rajas, F., & Mithieux, G. (2017). Gut-Brain Glucose Signaling in Energy Homeostasis. Cell Metab, 25(6), 1231-1242.

Vandenberghe, S., Moskal, P., & Karp, J. S. (2020). State of the art in total body PET. EJNMMI Physics, 7(1).

Vaz, S. C., Oliveira, F., Herrmann, K., & Veit-Haibach, P. (2020). Nuclear medicine and molecular imaging advances in the 21st century. Br J Radiol, 93(1110), 20200095.

Yang, J., Zhang, L. J., Wang, F., Hong, T., & Liu, Z. (2019). Molecular imaging of diabetes and diabetic complications: Beyond pancreatic β-cell targeting. Advanced Drug Delivery Reviews, 139, 32-50.

Zaccagna, F., Grist, J. T., Quartuccio, N., Riemer, F., Fraioli, F., Caracò, C., Gallagher, F. A. (2021). Imaging and treatment of brain tumors through molecular targeting: recent clinical advances. European Journal of Radiology, 109842.

1.-13. canceled

14. Method for computer-aided therapy for treating type 2 diabetes, insulin resistance and/or restoring glucose homeostasis in a subject in need, wherein a main frame is configured for performing the following steps:
a) receiving a subject profile created during a consultation of the subject by a MD at a first point of care, the subject profile comprising data useful for a therapy and the follow-up of the subject;
b) receiving a set of images associated with the subject, the set of images have been obtained at a second point of care, the set of images comprising a 3D computed tomography, CT, images of the abdomen of the subject including the pancreatic area and the portal vein of the subject; and a set of dynamic 3D positron emission tomography, PET, images of the abdomen after an intra veinous injection of a radiolabeled GLP-1r ligand, PET images comprising the positron imaging of the GLP-1r in the abdomen;
c) processing the PET and CT images for obtaining a 2D distribution of the quantitative density of the GLP-1r along the portal vein;
d) identifying a region with lower GLP-1r along the portal vein for which the values of the 2D distribution are less than a given threshold and for which the 2D distribution is less than or equal to a reference value; the threshold being based on the distribution of GLP-1r along the portal vein.

15. The method as claimed in claim 14, wherein the main frame is configured for performing the following steps
e) determining from the CT image and the 2D distribution, a first mesh representing the portal vein (PV);
f) determining from the CT image and the threshold a second mesh identifying the region with lower GLP-1r along the portal vein, the threshold being applied on the CT image;
g) determining from the first mesh and the second mesh a final 3D mesh wherein the second mesh is inside the first mesh.

16. The method as claimed in claim 15, wherein the main frame is configured for performing the step of:
h) determining a targeting solution by combining the final 3D mesh with the 3D CT image, the 3D CT image comprising several markers corresponding to externally applied fiducial marks the targeting solution comprising a 3D volumetric rendering comprising the 3D mesh of the portal vein coded with the density of the GLP-1r along the portal vein depending on the position relative to the threshold.

17. The method as claimed in claim 14, wherein the threshold is the mean—2*SE of the entire 2D distribution of the quantitative density of the GLP-1r along the portal vein.

18. The method as claimed in claim 14, wherein CT image and 3D volumetric rendering comprise images of external fiducial markers located on the subject skin whose positions in space XYZ are sufficient to identify the three dimensions of the surgical space.

19. The method as claimed in claim 14, wherein step c) comprises the sub steps of:
extracting from the CT image, a portal vein volume of interest including the portal vein;
obtaining from the PET images, a receptor density map quantifying indicator of the GLP-1r density in the abdomen;
obtaining from the receptor density map and the portal vein volume of interest a 3D image quantifying the GLP-1r density in the portal vein volume of interest;
obtaining a 2D distribution of the quantitative density of the GLP-1r density along the portal vein by projecting the 3D image along a center line of the second volume of interest.

20. The method as claimed in claim 14, wherein the region with lower GLP-1r for which the 2D distribution is less than the threshold, is a defective portal sensor.

21. The method as claimed in claim 14, wherein the main frame is configured for performing a step of extracting from the CT image a veinous branching location which is the location of the gastric, duodeno-pancreatic and splenic veins, a step of transferring to a first computing system of a MD such as a diabetologist or endocrinologist veinous branching location and the 2D distribution, so that a first computing system implements a step of displaying the 2D distribution along the veinous branching location to aid the MD to make a decision to process further the subject with a surgery procedure.

22. The method as claimed in claim 14, wherein the main frame is configured for performing a step of transferring the targeting solution to a third computing system at an abdominal surgeon/department.

23. The method according to as claimed in claim 14, wherein an abdominal surgeon department is connected to the mainframe, the abdominal surgeon department being configured for performing the steps of
i) providing at least one bioactive molecule upregulating the expression of GLP-1r, and
ii) locally administering said at least one bioactive molecule to the portal defective portal sensor identified as the mesh.

24. The method as claimed in claim 14, wherein a third point of care, preferably an abdominal surgeon/department is configured for performing a step of determining a customized targeting solution by obtaining, from the targeting solution a surgical procedure for accessing the area represented by the 3D mesh within the spatial CT reference.

25. The method as claimed in claim 24, wherein the surgical procedure includes laparoscopy, radiological or endoscopic solutions or a mixture of these.

26. The method as claimed in claim 18, wherein CT image and 3D volumetric rendering comprise at least three fiducial markers.

27. System for computer-aided therapy for treating type 2 diabetes, insulin resistance and/or restoring glucose homeostasis in a subject in need, comprising a main frame configured for performing the following steps:
a) receiving a subject profile created during a consultation of the subject by a MD at a first point of care, the subject profile comprising data useful for a therapy and the follow-up of the subject;
b) receiving a set of images associated with the subject, the set of images have been obtained at a second point of care, the set of images comprising a 3D computed tomography, CT, images of the abdomen of the subject including the pancreatic area and the portal vein of the subject; and a set of dynamic 3D positron emission tomography, PET, images of the abdomen after an intra veinous injection of a radiolabeled GLP-1r ligand, PET images comprising the positron imaging of the GLP-1r in the abdomen, the radiolabeled ligand is binding on GLP-1r;
c) processing the PET and CT images for obtaining a 2D distribution of the quantitative density of the GLP-1r along the portal vein;
d) identifying a region with lower GLP-1r along the portal vein for which the values of the 2D distribution are less than a given threshold and for which the 2D distribution is less than or equal to a reference value; the threshold being based on the distribution of GLP-1r along the portal vein.

28. The system as claimed in claim 27, comprising:
a first computer system configured to be accessed by a MD such as a diabetologist or endocrinologist;
a second computer system configured to be accessed by specialist of nuclear medicine;
at least one a third computer to be accessed by an abdominal surgeon/department;
the first, second and third computer being connected to the mainframe by means of a secure link.
